(19) United States
(12) Patent Application Publication
(10) Pub. No.: US 2007/0184464 A1
(43) Pub. Date: Aug. 9, 2007

(54) RNA probes

(76) Inventors: Azeddine Si-Ammour, Basel (CH); Todd Blevins, Colmar (FR); Frederick Meins JR., Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/548,925

(22) Filed: Oct. 12, 2006

Related U.S. Application Data
(63) Continuation of application No. 10/940,537, filed on Sep. 14, 2004, now abandoned.

(51) Int. Cl. C12Q 1/68; C12P 19/34

(52) U.S. Cl. 435/6; 435/91.2

(57) ABSTRACT

The invention is based in part on the generation of a double stranded RNA molecule substantially covering the whole transcribed region of a gene, and cleaving this using an RNA endonuclease to generate small RNA molecules which are already or may be subsequently labelled. The invention provides small labelled ribonucleic acid (RNA) fragments for use as probes to detect potentially small interfering ribonucleic acid (siRNA) fragments produced in vivo. The invention also provides uses of said small labelled RNA fragments and kits suitable for preparing said small labelled RNA fragments.

### FIELD OF THE INVENTION

The present invention relates to the provision of small labelled ribonucleic acid (RNA) fragments for use as probes to detect potentially small interfering ribonucleic acid (siRNA) fragments produced in vivo. The present invention also provides uses of said small labelled RNA fragments and kits suitable for preparing said small labelled RNA fragments.

### BACKGROUND TO THE INVENTION

RNA silencing, known as RNA interference (RNAi) in animals and post-transcriptional gene silencing (PTGS) in plants, is an important tool used to knockdown the expression of genes. In plants several methods can be used for this purpose (reviewed in Waterhouse & Helliwell, 2003). The most widely used method is the introduction of a hairpin structure covering a part or the whole coding region of the target gene. This construct is expressed using a strong promoter and a double-stranded RNA (dsRNA) is formed. This dsRNA is then cleaved by a Dicer-like RNAase III protein. Two classes of small RNAs (smRNAs) can be detected from the RNA-mediated silenced loci: 21-22nts or 24-26 nts. The small RNAs accumulation is crucial in the PTGS pathway and their detection by Northern analysis is important to show that the dsRNA template is indeed processed and is a potential target of RNA silencing and/or of antisense regulation.

However, in some instances it can be difficult to design probes for the detection of such small RNAs. For example, using the method described in Glazov et al., 2003, to detect small RNAs, the present inventors have found that the RNA probes, when used, generate a high background signal, which is undesirable and can lead to difficulties in interpreting results. Alternatively, if labelled DNA oligonucleotide probes are used, the background is low, but no signal could be detected, presumably because the oligonucleotide probes were not covering the whole coding region of the gene being restricted to siRNA fragments and thus does not provide sufficiently high sensitivity to detect low-abundance smRNAs. To counter this it is possible to use a number of 5′-labelled DNA oligonucleotides as probes, covering the whole silenced region, but it is to be appreciated that this is extremely expensive.

The most widely used method to detect small RNAs is based on the method of Hamilton & Baulcombe, 1999. This technique is based on an in vitro transcription method to generate a long radiolabelled transcript which is thereafter hydrolysed to generate fragments averaging 50 nucleotides in length. However, this method has a number of disadvantages: it is time consuming and the exposure to radioactivity is very high. The radiolabelled probes are not stable and decay on storage. Moreover, the hydrolysis of the probes is not always optional, resulting in very high background radioactivity and lack of reproducibility.

It is amongst the objects of the present invention to obviate and/or mitigate at least one of the aforementioned disadvantages.

It is an object of the present invention to provide a simple method to prepare small labelled RNA probes for use in detecting small single stranded RNA fragments generated in vivo, which may serve to function as siRNA in RNA silencing and to transcriptional gene silencing or specific modifications in chromatin that may be important for epigenetic regulation (see Dykxhoom et al., 2003; Ekwall, 2004a; Ekwall, 2004b, Ichim et al., 2004; Kawasaki & Taira, 2004).

### SUMMARY OF THE INVENTION

The present invention is based in part on the generation of a double stranded RNA molecule substantially covering the whole transcribed region of a gene, and cleaving this using an RNA endonuclease to generate small RNA molecules which are already or may be subsequently labelled.

Thus, in a first aspect there is provided a method of generating small labelled ribonucleic acid (RNA) fragments, comprising the steps of:
a) providing a large unlabelled double stranded RNA fragment;
b) cleaving said large double stranded RNA fragment using an RNA endonuclease in order to generate small RNA fragments; and
c) labelling said small RNA fragments in order to generate said small labelled RNA fragments.

In a second aspect there is provided a method of generating small labelled RNA fragments, comprising the steps of:
a) providing a large labelled double stranded RNA fragment; and
b) cleaving said large double stranded RNA fragment using an RNA endonuclease in order to generate said small labelled RNA fragments.

In accordance with the first and second aspects of the present invention, the large unlabelled or labelled double stranded RNA fragment may be prepared by in vitro transcription of a DNA fragment. Generally, this will be carried out by first cloning an appropriate DNA fragment into an appropriate cloning vector which is capable of transcribing sense and anti-sense RNA molecules from the cloned DNA fragment.

Typically the appropriate DNA fragment may comprise the whole coding region of a gene being studied in gene silencing experiments. Gene silencing involves the generation of siRNA and it is a purpose of the present invention to detect such siRNA fragments produced in vivo. The procedure by which siRNAs are generated is not the subject of the present invention and has been discussed in detail in the art and is well known by a skilled addressee (see for example Waterhouse & Helliwell, 2003). By employing a DNA fragment comprising the whole coding region of a gene being studied in gene silencing experiments, the present invention is capable of generating small labelled RNA fragments covering the entire coding region. This is advantageous, as it is often not known how an mRNA fragment is specifically broken down to generate siRNAs.

If it is desired to produce the large labelled double stranded RNA fragment in accordance with the second aspect of the present invention, in vitro transcription may simply be carried out in the presence of labelled deoxynucleotide triphosphates (dNTPs), which may be labelled for example with a radiolabel, chemiluminescent label, or fluorescent label as known to those skilled in the art (see for example Sambrook et al., 2000). It will be appreciated that it is possible to label both strands by including an appropriately labelled dNTP in both transcription reactions in order to generate labelled sense and anti-sense RNA, or alternatively only one strand may be labelled during the in vitro transcription reaction by using a labelled dNTP in only one transcription reaction, in order to generate a labelled sense or anti-sense RNA fragment.

In order to generate the small RNA fragments, the large double stranded RNA fragment is cleaved using an RNA endonuclease such as Dicer available from Gene Therapy Systems (Gene Therapy Systems, San Diego, USA) or Dicer-like enzyme RNase III enzyme (available from Stratagene, LaJolla, USA; Ambion, Inc., Austin, USA; and Invitrogen, Paisley, UK). By incubating the large labelled or unlabelled double stranded RNA fragment with the RNA endonuclease, using appropriate conditions as described by the manufacture, the large double stranded RNA fragment is cleaved, thereby generating small RNA fragments of approximately 15-30 nucleotides in length. If appropriate, the RNA endonuclease may thereafter be removed from the small RNA fragments, using spin chromatography with, for example Sephadex G-25 (Amersham, UK). Thus, it is to be understood that the term “small RNA fragment” as used in the present invention relates to fragments of less than about 30 nucleotides in length, such as about 21-25 base pairs in length.

It will be understood to the skilled addressee, that said small RNA fragments will be labelled, if the in vitro transcription reaction was carried out in the presence of a labelled dNTP. However, if the in vitro transcription reaction is carried out in the absence of labelled dNTPs, a large unlabelled double stranded RNA fragment is generated. In such cases, said large unlabelled double stranded RNA fragment is cleaved to generate small unlabelled RNA fragments, and it is necessary to subsequently label the so generated small RNA fragments.

Labelling of the small unlabelled RNA fragments may easily be carried out using for example a phosphatase/kinase reaction well known to the skilled addressee and described, for example in Sambrook et al., 2000. However, in summary the small unlabelled RNA fragments are generally dephosphorylated using alkaline phosphatase in order to generate 5′-hydroxyl groups. Thereafter the small dephosphorylated RNAs may be labelled with, labelled dNTP using for example a polynucleotide kinase. Typically this may be carried out using [γ-³²P]dATP or alternatively using fluorescently labelled, or chemiluminescently labelled, dNTPs as known in the art (e.g. digoxygenin, biotin, Cy3-, or Cy5-dNTPs and the like), in order to generate small labelled RNA fragments.

In this manner, small RNA fragments may be generated covering the entire coding region of the original DNA fragment which is transcribed into double stranded RNA and the small labelled RNA fragments produced there from may be used to check for the accumulation of siRNAs as generated by an in vivo silencing pathway, by Northern analysis as known in the art (see Sambrook et al., 2000).

In a further aspect there is provided a kit for use in generating small labelled ribonucleotide acid (RNA) fragments, the kit comprising a cloning vector for use in generating a large double stranded RNA fragment; and an RNA endonuclease which is capable of cleaving said large double stranded RNA fragment in order to generate small labelled or unlabelled RNA fragments.

It is to be understood that the kit may comprise further reagents such as the reagents necessary for carrying out an amplification reaction, such as PCR; a labelled dNTP for labelling said large double stranded RNA fragment or small RNA fragments: one or two RNA polymerases for effecting in vitro transcription of the cloned DNA fragment and optionally reagents therefore; and/or alkaline phosphatase and a polynucleotide kinase for dephosphorylating said small unlabelled RNA fragments and subsequently labelling these with an appropriately labelled dNTP. The above mentioned kit may contain instructions.

In a further aspect, there is provided use of small labelled RNA fragments as probes for detecting siRNA fragments produced in vivo. Typically the small labelled RNA fragments will be used as probes in a Northern blot. The skilled addressee is well aware of how to carry out a Northern blot experiment, but details are found in Sambrook et al., 2000.

### BRIEF DESCRIPTION OF THE DRAWINGS

FIG. 1 shows a scheme in accordance with one embodiment of the present invention; and

FIG. 2 shows a small Northern blot using small radiolabelled RNA fragments prepared according to the present invention.

### DETAILED DESCRIPTION OF THE INVENTION

The present invention will now be described further in more detail and by way of example and with reference to the figures which show:

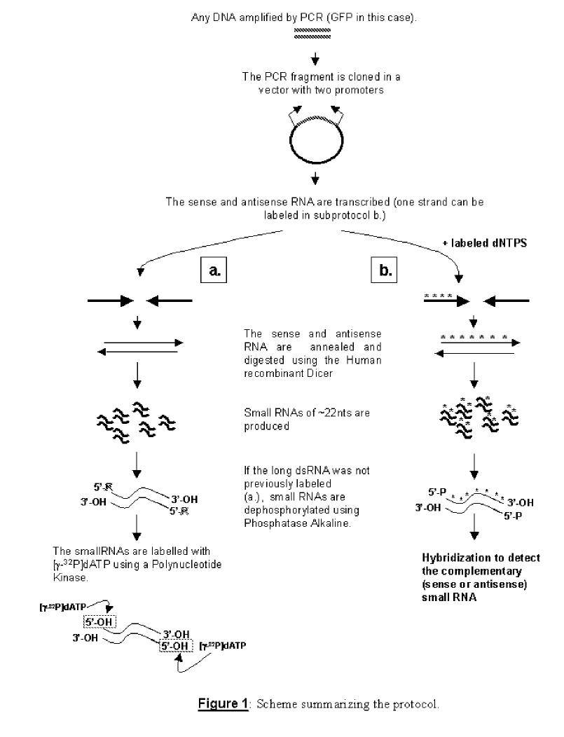

FIG. 1 shows a scheme in accordance with one embodiment of the present invention. In summary the scheme shows a method suitable for generating small unlabelled RNA fragments from a large unlabelled RNA fragment and subsequently labelling said small unlabelled RNA fragments. The steps which are carried out, are as follows:
a) a gene/coding region of interest is first amplified using polymerase chain reaction (PCR) to generate an amplified DNA fragment;
b) the amplified DNA is cloned into an appropriate cloning vector using techniques well known in the art for cloning PCR products (see for example Sambrook et al, 2000). For example, TA cloning vectors as known in the art, may be employed;
c) once cloned, the DNA fragment is transcribed using appropriate RNA polymerases and their promoters flanking the multiple cloning region into which the gene has been inserted. Many vectors are known in the art which are capable of generating sense and anti-sense RNA strands such as the TOPO and Gateway vectors supplied by Invitrogen, pGEM vectors supplied by Promega and pBlueScript vectors provided by Stratagene. All suitable vectors possess two promoters at either end of the cloning region and by using an RNA polymerase appropriate for each promoter, it is possible to transcribe either the sense or anti-sense RNA using this promoter. The techniques for carrying out such in vitro transcription are well known to those skilled in the art and again are described in Sambrook et al., 2000 or in the protocols provided by the relevant manufacture;
d) the transcribed sense and antisense RNA strands are allowed to anneal, thereby generating said large unlabelled RNA fragment;
e) the large unlabelled RNA fragment is thereafter digested using human recombinant Dicer (Genetic Therapy Systems, Inc., San Diego, US), according to manufacturer's instructions, in order to generate small RNA fragments of about 22-25 nucleotides in length; and
f) the small RNA fragments are dephosphorylated using alkaline phosphatase and subsequently labelled with [γ-³²P] dATP using a polynucleotide kinase (FIG. 1a).

The so generated small labelled RNA fragments may thereafter be used in Northern experiments, known to those skilled in the art, to identify whether or not the same gene/coding sequence is processed in vivo to generate potentially small interfering RNAs.

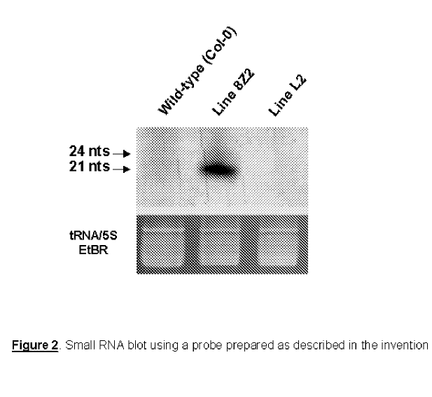

FIG. 2 shows a small RNA Northern blot using radioactively labelled small RNA probes prepared according to the present invention. The accumulation of GFP small RNAs, a strong band at 21 nucleotides (nts) and a fainter band at 24 nts, in the silenced GFP reporter line 8Z2 (Glazov et al., 2003) is easily identified. As expected, the wild-type plants Col-0 and the high GFP expressing line 2 (Glazov et al., 2003) did not show any accumulation of GFP smRNAs. Briefly, 20 μg of smRNAs were loaded on a 15% polyacrylamide/8M urea gel. The gel was electroblotted on a HybondN+ membrane (Amersham). The membrane was prehybridized for 1 hour in the Ultrahyb-oligo buffer (Ambion). For the preparation of the small RNA probe, 5 μg of in vitro transcribed GFP dsRNA (5 μg of sense GFP RNA annealed with 5 μg of antisense GFP RNA) were digested using the Human Recombinant Dicer (GTS) according to the manufacturer's instructions. The digestion product was purified with G-25 spin columns (Amersham). The small RNAs were further dephosphorylated using the Shrimp Alkaline Phosphatase (SAP, Roche) and purified again with the G-25 spin columns. At this step the concentration of the small RNAs is estimated in pmoles/μl and can be kept in −20° C. for future experiments. Finally, 20 pmoles of the Diced GFP small RNAs were labeled at the 5′end with 20 pmoles of [γ-³²P]dATP using Polynucleotide Kinase (PNK, Roche). The probe was purified with G-25 spin columns, denatured at 95° C. and added to the prehybridization buffer (UltraHyb-oligo, Ambion). The hybridisation was performed for at least 16 hours and the membrane washed according to the manufacture. The signal was detected either using a phosphoimager screen or using a Biomaz MR X-ray film.

### REFERENCES

Waterhouse, P. M. & Helliwell, C. A. (2002) “Exploring Plant Genomes by RNA-induced Gene Silencing.” Nature Rev. Genet., 4, 29-38.

Glazov, E.; Phillips, K.; Budziszewski, G. J.; Meins, F. & Levin, J. Z. (2003) “A gene encoding an RNAse D exonuclease-like protein is required for post-transcriptional silencing in Arabidopsis.” Plant J., 35, 342-349

Hamilton, A. J. & Baulcombe, D. C. (1999) “A Species of Small Antisense RNA IN posttranscriptional Gene Silencing in Plants.” Science, 286, 959-952.

Dykxhoorn, D. M., Novina, C. D. & Sharp P. A. (2003) Nat. Rev. Mol. Cell Biol., 6, 457-467.

Ekwall, K. (2004a) “The RITS complex-A direct link between small RNA and heterochromatin.” Mol. Cell, 13, 304-305.

Ekwall, K. (2004b) “The roles of histone modifications and small RNA in centromere function.” Chromosome Res., 12, 535-42.

Ichim, T. E.; Li, M.; Qian, H; Popov, I. A.; Rycerz, K.; Zheng, X.; White, D.; Zhong, R. & Min, W. P. (2004) “RNA Interference: A Potent Tool for Gene-Specific Therapeutics.” American Journal of Transplantation, 4, 1227-1236.

Kawasaki, H. & Taira, K. (2004) Nature, 431, 211-2177

Sambrook, J.; Fritsch, E. F.; & Maniatis, T. Molecular Cloning: A Laboratory Manual. (Cold Spring Harbor Laboratory Press, 2000).

What is claimed:

1. A method of generating small labelled ribonucleic acid (RNA) fragments, comprising the steps of:
(a) providing a large unlabelled double stranded RNA fragment;
(b) cleaving said large double stranded RNA fragment using an RNA endonuclease in order to generate small RNA fragments; and
(c) labelling said small RNA fragments in order to generate said small labelled RNA fragments.

2. The method according to claim 1 wherein the large unlabelled double stranded RNA fragment is prepared by in vitro transcription of a DNA fragment.

3. The method according to claim 2 wherein the DNA fragment comprises a whole transcribed region of a gene.

4. A method of generating small labelled RNA fragments, comprising the steps of:
(a) providing a large labelled double stranded RNA fragment; and
(b) cleaving said large double stranded RNA fragment using an RNA endonuclease in order to generate said small labelled RNA fragments.

5. The method according to claim 4 wherein the large unlabelled double stranded RNA fragment is prepared by in vitro transcription of a DNA fragment.

6. The method according to claim 5 wherein the DNA fragment comprises a whole transcribed region of a gene.

7. The method according to claim 4 wherein said in vitro transcription is carried out in the presence of labelled deoxynucleotide triphosphates (dNTPs) in order to generate said large labelled double stranded RNA fragment.

8. The method according to claim 7 wherein one or both strands of said large double stranded RNA fragment are labelled.

9. The method according to claim 1 wherein the RNA endonuclease is the Dicer or Dicer-like enzyme.

10. The method according to claim 4 wherein the RNA endonuclease is the Dicer or Dicer-like enzyme.

11. The method according to claim 1 wherein the small RNA fragments are labelled using a phosphatase/kinase reaction.

12. The method according to claim 1 wherein said small labelled RNA fragments are labelled with a radio-, chemiluminescent-, or fluorescent-label.

13. The method according to claim 4 wherein said small labelled RNA fragments are labelled with a radio-, chemiluminescent-, or fluorescent-label.

14. A kit for use in generating small labelled ribonucleotide fragments, the kit comprising a cloning vector for use in generating a large double stranded RNA fragment: and an RNA endonuclease which is capable of cleaving said large double stranded RNA fragment in order to generate small labelled or unlabelled RNA fragments.

15. The kit according to claim 14 additionally comprising at least one of the following:
reagents necessary for carrying out an amplification reaction, such as PCR; a labelled dNTP for labelling said large double stranded RNA fragment or small RNA fragments: one or two RNA polymerases for effecting in vitro transcription of the cloned DNA fragment and optionally reagents therefore; and/or alkaline phosphatase and a polynucleotide kinase for dephosphorylating said small unlabelled RNA fragments and subsequently labelling these with an appropriately labelled dNTP.
